(19) United States
(12) Patent Application Publication
(10) Pub. No.: US 2007/0049739 A1
(43) Pub. Date: Mar. 1, 2007

(54) Method and system for extracting blood-derived growth factors

(75) Inventors: Karen S. Troxel, Warsaw, IN (US); Felipe Palacios, Pearland, TX (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/214,661

(22) Filed: Aug. 30, 2005

(51) Int. Cl. C07K 14/475

(52) U.S. Cl. 530/399; 530/412

(57) ABSTRACT

Methods for extracting heparin-binding growth factors from whole blood comprising contacting whole blood with a heparin-conjugated system to immobilize a conjugated fraction comprising the heparin-binding growth factors; separating a non-conjugated fraction from the system; and releasing the heparin-binding growth factors are provided. Kits include a heparin-conjugate immobilized to the surface of a substrate; and a device to withdraw whole blood from a human or animal subject. Methods of promoting tissue health with the growth factors are also provided.

### INTRODUCTION

This invention relates to methods for isolating growth factors from whole blood.

Blood-derived growth factors are useful in several applications including wound healing, orthopedic bone defect repair, bone fixation and implantation procedures, plastic surgery, connective tissue repair, periodontal surgery, and to create new blood vessels in previously damaged tissues.

Acquiring blood-derived growth factors by current techniques is a multi-step process that can extended periods of time, up to days or weeks, to complete. First, the blood is drawn from a subject or suitable blood is located and received from a donor facility. Blood is then sent to a laboratory where blood is processed into homogenous fractions. Homogenizing the blood fractions is complex and requires that the whole blood is heated, precipitated using salts, filtered, contacted with receptor proteins, or subjected to a variety of pressure treatments. It requires a high level of user input through repetitive processing, discarding unwanted fractions, and processing cycle. Aside from transfer delays between the originating facility and the laboratory, homogenization is the most time consuming steps required to extract growth factors from blood. Even the simplest of these techniques require at least a partial purification of the blood prior to separation of growth factors. Homogenizing is timely, costly, inefficient, and can be extremely inconvenient for health care or other facilities where it is desirable to rapidly extract growth factors from the blood.

It would be advantageous to provide a method for separating growth factors from the whole blood that is simple, completed without the need for homogenizing fractions, is cost effective, and provides adequate isolation and concentration of growth factors.

### SUMMARY

The present invention provides methods for extracting heparin-binding growth factors from whole blood comprising: contacting whole blood with a heparin-conjugated system to immobilize a conjugated fraction comprising the heparin-binding growth factors; separating a non-conjugated fraction from the system; and releasing the heparin-binding growth factors.

Kits are also provided comprising a heparin-conjugate immobilized on a substrate and a device to withdraw whole blood from a human or animal subject.

Methods of promoting tissue health are also provided comprising contacting whole blood with a heparin-conjugated system to separate the whole blood into a conjugated fraction and a non-conjugated fraction; separating the non-conjugated fractions to provide growth factors; and applying the growth factors to the tissue.

The methods and kits of this invention provide benefits over methods and devices among those known in the art. Such benefits may include one or more of: efficiently extracting growth factors from whole blood, rapidly providing growth factors for autologous donation, providing an adequate concentration of growth factors, and providing growth factors useful promoting tissue health. Further areas of applicability will become apparent from the detailed description provided hereinafter. It should be understood that the detailed description and specific examples, while indicating preferred embodiments of the invention, are intended for purposes of illustration only and are not intended to limit the scope of the invention.

### DESCRIPTION

The present invention provides methods and kits to extract growth factors from whole blood. The following definitions and non-limiting guidelines must be considered in reviewing the description of this invention set forth herein.

The headings (such as “Introduction” and “Summary”) and sub-headings (such as “Treatment of Whole Blood”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being a “therapeutic” ingredient or “promoting tissue health”) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. As referred to herein, all compositional percentages are by weight of the total composition, unless otherwise specified.

As used herein, the word “include,” and its variants, is intended to be non-limiting, such that recitation of items in a list is not to the exclusion of other like items that may also be useful in the materials, compositions, devices, and methods of this invention.

Methods of Extracting Growth Factors

Various methods of the present invention facilitate extraction of heparin-binding growth factors from whole blood. Methods include contacting whole blood with a heparin-conjugated system to immobilize heparin-binding growth factors that are bound to the heparin as “conjugated fractions”. Next, the “non-conjugated” fractions are separated from the system. The heparin-binding growth factors are then released from the heparin-conjugated system. While, in various embodiments, the methods are single-step and/or performed in a single-container, discussion of the various operations may be divided into the following sections for clarity.

### Treatment of Whole Blood

As used herein, “whole blood” includes all cellular and plasma components of blood from human or other animal subjects, such as pigs and cows. Whole blood includes blood fractions, such as plasma, blood cells, blood factors, blood related proteins, and growth factors. Whole blood is either freshly removed from a donor or it has been previously removed and stored, such as from a blood bank.

The whole blood is preferably treated to activate the platelets and release the growth factors. The platelet endothelium layer is disrupted and platelet contents are released into the plasma and/or other surrounding fluid, thereby providing an “activated” platelet. Platelet activation can be achieved by application of shear force, chemical treatment, and combinations thereof. Preferred chemical treatments include contacting the whole blood with collagen, thrombin, thromboxane, adenosine diphosphate (ADP), epinephrine, and mixtures thereof. In an embodiment where chemical treatment is used, the whole blood is mixed with the activating chemical by coating the inside of a receptacle for the blood (such as a syringe, blood bag, test tube) with the solution containing the activating agent or by adding the activating agent to the receptacle before or after the addition of the whole blood.

Chemical interactions between platelet cell contents and surrounding fluids trigger the conversion of prothrombin in the blood to thrombin. The thrombin in turn converts fibrinogen to fibrin that assembles into fibrils to develop a blood clot. The presence of thrombin in the system activates nearby platelets and the clot expands. Clotting is preferably minimized or eliminated in various embodiments of the present invention because the growth factors may become trapped in the clot or the blood clot size may hinder the separation process.

To prevent clotting, the whole blood solution having the activated platelets is preferably treated with an anticoagulant. “Anticoagulants” include substances that slow, suppress, or prevent clotting including lithium heparin, ammonium heparin, sodium heparin, ethylene diamine tetraacetic acid, acid citrate dextrose, sodium citrate, citrate phosphate dextrose, sodium fluoride, sodium oxalate, potassium oxalate, lithium oxalate, sodium iodoacetate, lithium iodoacetate, and combinations thereof.

In various embodiments, additional agents are added to the whole blood with activated platelets to facilitate removal of the growth factors. Agents are advantageously added at the point of withdraw of blood (coated inside of the syringe, for example) or may be added later. In a preferred embodiment, such agents include highly glycosylated peptides, proline-rich peptides, and detergents, and anticoagulants or platelet activators, in addition to those listed above. The highly glycosylated peptides such as glycosylated proline-rich proteins (GPRP) are proteases that prevent various proteins from forming of fibrin clots. The detergents promote lysis of the platelets and break up the material that may adhere to the growth factors. Useful detergents have appropriate strength to lyse cells and include anionic, cationic, nonionic, and zwitterionic detergents. Preferred detergents are sodium-dodecyl sulfate, sodium deoxycholate, polyglycol ether surfactants, and combinations thereof.

### Growth Factors and Heparin Affinity

“Growth factors” as used herein refers to the class of polypeptides that stimulate target cells to proliferate, differentiate, or organize in developing tissues. Examples of growth factors include platelet-derived growth factor (PDFG), insulin-like growth factor (IGF-I, IGF-II), interleukin-I, interferon, transforming growth factor beta (TGF-α), epidermal growth factor (EGF), acidic and basic fibroblast growth factors (αFGF, βFGF), connective tissue growth factor (CTGF), tumor necrosis factor (TNF), platelet-derived angiogenesis factor (PDAGF), nerve growth factor (NGF), and platelet-derived epidermal growth factor (PDEGF). Growth factors have binding sites that stimulate a surrounding event within the cell or have an affinity to certain classes of compounds. Depending on the signal or the class of compound, each growth factor has different binding sites.

Many growth factors share a binding site affinity for heparin. Heparin is a heterogeneous group of straight-chain anionic mucopolysaccharides called glycosaminoglycans. The sugars in the heparin are joined by glycosidic linkages, forming polymers of various sizes. “Heparin” as used herein includes unfragmented (naturally occurring) heparin, low molecular weight heparins, heparan, heparinoids, heparin related compounds, salts thereof, esters thereof, fatty acid conjugates thereof, and combinations thereof. The heparin binding site for each of the growth factors is particular to the genetic sequence of the particular growth factor and the species of the subject. While not intending to be bound by a particular theory, it is believed that the sulfate and carboxylate groups of the heparin interact with the binding sites of the growth factors to adhere the growth factors to the heparin or adhesion results from the heparin coiling around the binding site.

The heparin is immobilized using a heparin-conjugated system to isolate the growth factors. In various embodiments, the heparin-conjugated system is selected from heparin-conjugated beads, heparin-affinity columns, heparin-conjugated plates, heparin chromatography, and combinations thereof.

Beads among those useful in the present invention include sepharose, agarose, polystyrene, or magnetic beads. In various embodiments, the heparin is immobilized on the bead substrate by applying a liquid solution containing heparin to the substrate and then volatilizing the solution, adsorbing heparin to the substrate, or cross-linking the heparin with the substrate. The beads are solid and have a heparin coating on their outer surface and/or are porous and trap growth factors within heparin coated pores. The bead and pore size are preferably sufficient to accommodate the heparin and trap the growth factors while still allowing passage of the non-conjugated compounds through the system. As discussed later herein, the selection of the bead type will determine the technique used to release the growth factors from the system. Heparin-conjugated beads are used in a ratio of from about 1:4 to about 1:30 volume to volume with the whole blood.

Heparin-affinity columns are columns packed with the heparin-conjugated beads. The amount of beads in the column is determined based on the relative levels of whole blood to other agents (anticoagulants, for example), extent of cell lysis, desired end concentration, and the desired time of the extraction. Preferably, beads in the column are sepharose or agarose beads.

Heparin-conjugated plates are created by mixing polystyrene and heparin and then forming the mixture on the bottom of a plate such as a Petri dish. The amount of blood added to a heparin-conjugated plate depends on the binding potential of the plate. In embodiments where the plate contains a high amount of heparin (over 15% by weight), more activated whole blood can be added to the plate as there is an increased number of potential binding sites for the growth factors. Conversely, in embodiments where the plate has a low amount of heparin (less than 5% by weight), less activated whole blood can be added as there are limited binding sites. The amount of activated whole blood added to the heparin-conjugated plate also depends on the size of the plate.

As stated, blood can be withdrawn from the subject and deposited directly into any of these systems. The heparin-conjugated system remains in contact with the blood solution for a period of time and under conditions sufficient to promote adhesion of the growth factors to the heparin-conjugated system. Contact time may range from minutes to hours and is preferably from about 30 minutes to about 2 hours. The respective container or tube is rotated, centrifuged, incubated, or cooled to provide adherence of the growth factors to the heparin-conjugate and to adequately isolate the growth factors.

### Releasing Growth Factors

Releasing the growth factors bound to the heparin-conjugated system is performed using techniques well known in the art. Suitable techniques include washing, centrifuging, or gravity filtering the whole blood solution, and combinations thereof. The particular technique(s) employed depends on the type of immobilization system used.

In embodiments using heparin-conjugated beads, the beads settle by gravity or are centrifuged to form a pellet. Centrifugation occurs at from about 500 revolutions per minute (RPM) up to about 5000 RPM. The RPM may be increased or decreased to any speed depending on the size of the beads and the spin potential or speed limitations of the centrifugation apparatus. The supernatant is decanted leaving the beads having the conjugated growth factors adhered thereto. The beads are washed with a suitable buffer. The buffer is preferably a phosphate buffered saline (PBS) solution free of calcium and magnesium, having a neutral pH of from about 6 to about 8, preferably about 7.4. The buffer removes the non-conjugated blood material.

In embodiments utilizing the heparin-conjugated affinity column, the column is washed with buffer to remove the anticoagulant, residual detergent, or any other accompanying solutions. A preferred buffer is a phosphate buffered saline. The growth factors are eluted using a high salt solution or a competitive binding material that has a higher affinity towards the heparin-conjugated system than the growth factors. In preferred embodiments, a heparin interacting protein or heparin-sulfate interacting protein (both referred to as HIP), or peptides thereof, having a greater affinity for heparin than the growth factors, displace the growth factors causing them to be released from the column and elute into the buffer. The eluted buffer fraction containing the growth factors is isolated and may be combined with other isolated growth factor containing fractions to concentrate the growth factors.

In embodiments where a heparin-conjugated plate is used, the plate preferably is washed with buffer to remove the non-conjugated fraction including any anticoagulants, residual detergents, or other accompanying solutions. The growth factors are removed from the plate using the high salt solution, such as urea, or competitive binding material as described above.

Kits

Kits of various embodiments comprise: a heparin-conjugate immobilized to the surface of a substrate and a device to withdraw whole blood from a human or animal subject. Heparin-conjugate immobilized systems include the heparin-conjugated beads, heparin-conjugated columns, and heparin-conjugated plates disclosed earlier herein. These immobilized systems contain an amount of heparin sufficient to bind the growth factors in whole blood.

In various embodiments, components for user fabrication of the heparin-conjugate system are included in the kit. Such components include agarose beads, Petri dishes, polystyrene, and heparin. Such user fabrication may provide a high level of user control, especially with the preparation of the heparin-conjugated plates.

In various embodiments, the kit further includes agents useful in the single-step processing of whole blood, including anticoagulants, glycosylated or proline-rich peptides, detergents, and platelet activators, such as those listed earlier herein. In various embodiments, whole blood is also in the kit. The blood may be from a donor source of the same or a different species as the intended recipient.

In various embodiments, instructions for using the kit are also provided. The instructions detail the use of whole blood and contacting the whole blood with the heparin-conjugated system. In embodiments where the heparin-conjugated system is fabricated, the instructions detail the specific amounts of heparin to coat onto the substrate along with the amount of contact time needed between the whole blood with the heparin-conjugated system. For example instructions for use with heparin-conjugated metal beads could recite the use of a magnet to precipitate the beads. For various systems, the instructions also detail the amounts of whole blood needed to obtain determined concentrations of growth factors based on criteria such as donor whole blood species, donor age, amount of time the whole blood was stored, and the desired growth factors for extraction, for example.

Methods of Promoting Tissue Health

In various embodiments, the present invention provides methods of promoting tissue health. “Promoting tissue health” refers to repair of an unhealthy or damaged tissue, maintenance of healthy tissues, and preventative measures against disease or defects in healthy or damaged tissue.

Whole blood is contacted with the heparin-conjugated system to separate the whole blood into a conjugated fraction and a non-conjugated fraction. The non-conjugated fraction is removed leaving the growth factors. The growth factors are isolated and concentrated, according to various techniques detailed above, and the growth factors are applied to the tissue.

Growth factors can be applied to the area surrounding a wound or surgically remodeled bone or cartilage or the growth factors can be coated on an implant. For example, the growth factors extracted according to various embodiments, are coated onto an implant, such as a prosthetic knee or a skin graft. The growth factor coating is useful for resorbable and non-resorbable implants. In embodiments using a resorbable implant, as the implant degrades, the growth factors lure the healthy growing tissue into the dissolved portions, thereby providing a supporting scaffold or structure throughout the duration of the tissue ingrowth.

Other compounds may also be added to the site where tissue health is promoted such as vitamins, minerals, pluripotent cells, multipotent cells, inorganic materials, amino acids, gelatin, naturally occurring or synthetic therapeutic drugs, proteins, enzymes, and therapeutic agents. Therapeutic agents include anti-inflammatory agents, additional growth factors, nutrient factors, and mixtures thereof. See U.S. Pat. No. 6,086,863, Ritter, et al., issued Jul. 11, 2000, and U.S. Pat. No. 6,180,606, Chen, et al., issued Jan. 30, 2001.

The present invention is further illustrated through the following non-limiting examples.

### EXAMPLE 1

50 cubic centimeters (cc) of whole blood is extracted in the presence of anticoagulant citrate dextrose solution, glycosylated proline-rich protein (GPRP) peptides, and the platelet activator thrombin using a 60 cc syringe. The GPRP peptides prevent the formation of fibrin clots upon activation of platelets. The resulting blood solution is passed through a heparin-conjugated affinity chromatography column and washed thoroughly with phosphate buffer solution (PBS) to remove the unbound proteins and the anticoagulant solution. The growth factors bound to the heparin-affinity column are eluted using 5 milliliters (ml) of the high-salt solution of urea.

### EXAMPLE 2

50 cc of whole blood is extracted in the presence of anticoagulant citrate dextrose solution, GPRP peptides, and the platelet activator collagen using a 60 cc syringe. The blood solution is loaded into a conical tube containing 5% volume/volume of heparin-conjugated sepharose beads. The tube is tightly capped and shaken for one hour at room temperature using a rotation mixer. The blood solution is centrifuged at 1,000 RPM for five minutes to precipitate the beads. The supernatant is decanted and the beads are washed with 50 mL of PBS, pH: 7.4. A second centrifugation is performed at 1,000 RPM for five minutes. The PBS is decanted and 5 ml of PBS containing excess of HIP peptide is added to the beads and incubated for 15 minutes at room temperature with moderate shaking. The resulting solution is then centrifuged at 1,000 RPM for five minutes. The supernatant containing the growth factors is carefully collected using a syringe.

### EXAMPLE 3

50 cc of whole blood is extracted in the presence of 5 mL anticoagulant citrate dextrose solution and the platelet activator thrombin using a 60 cc syringe. The detergent sodium dodecyl sulfate (SDS) is added to the blood solution to 1.0% volume/volume and shaken for 15 minutes at room temperature using a rotation mixer. The detergent promotes the lysis of cells and platelets in the blood releasing all the growth factors and other substances. The resulting blood solution is then passed through a heparin-conjugated affinity chromatography column and washed thoroughly with phosphate buffered saline to remove the anticoagulant solution and the residual detergent. The growth factors bound to the heparin-affinity column are eluted using 5 ml of PBS containing an excess of HIP peptide. The eluted solution contains the growth factors.

### EXAMPLE 4

Growth factors are prepared according to Example 3. The growth factors are applied to a skin-graft site on a burn victim. The newly grafted skin covers the burn and there is expedited healing and ingrowth of healthy skin tissue.

### EXAMPLE 5

50 cc of whole blood is extracted in the presence of 5 mL anticoagulant citrate dextrose solution and the platelet activator thrombin using a 60 cc syringe. The detergent sodium dodecyl sulfate (SDS) is added to the blood solution to 1.0% volume/volume and 5% volume/volume of heparin-conjugated agarose beads are added. The tube is tightly capped and shaken for one hour at room temperature using a rotation mixer. The blood solution is then centrifuged at 1,000 RPM for five minutes to precipitate the beads. The supernatant is decanted and the beads are washed with 50 ml of PBS 1X pH: 7.4. A second centrifugation is performed at 1,000 RPM for five minutes. The PBS is decanted and 5 ml of PBS 1X containing excess of HIP peptide is added to the beads and incubated for 15 minutes at room temperature with moderate shacking. The resulting solution is centrifuged at 1,000 RPM for five minutes and the supernatant containing the growth factors is carefully collected using a 5 ml syringe without disturbing the beads pellet.

### EXAMPLE 6

The growth factor-rich solution prepared in Example 5 is combined with fibrin glue. The resulting solution is layered onto the resorbable matrix overlying an acromioclavicular ligament implant.

What is claimed is:

1. A method for extracting heparin-binding growth factors from whole blood comprising:
contacting whole blood with a heparin-conjugated system to immobilize the heparin-binding growth factors;
separating a non-immobilized fraction from the system; and
releasing the heparin-binding growth factors.

2. A method according to claim 1, wherein the heparin-conjugated system is selected from the group consisting of heparin-conjugated beads, heparin-affinity columns, heparin-conjugated plates, heparin chromatography, and mixtures thereof.

3. A method according to claim 1, wherein the heparin is selected from the group consisting of unfragmented heparin, low molecular weight heparins, heparan, heparinoids, heparin related compounds, salts thereof, esters thereof, fatty acid conjugates thereof, and combinations thereof.

4. A method according to claim 1, further comprising activating platelets in the blood prior to the contacting, wherein the activating is selected from the group consisting of applying shear force, chemical treatment, and combinations thereof.

5. A method according to claim 4, wherein the activating comprises a chemical treatment comprising contacting the whole blood with collagen, thrombin, thromboxane, ADP, epinephrine, and mixtures thereof.

6. A method according to claim 1, further comprising purifying the conjugated fraction.

7. A method according to claim 1, wherein the separating the non-immobilized fraction is performed using a treatment selected from the group consisting of washing, centrifuging, gravity filtering, and combinations thereof.

8. A method according to claim 7, wherein the treatment comprises eluting a washing comprising competitive solution through the heparin-conjugated system.

9. A method according to claim 8, wherein the competitive solution is selected from the group consisting of HIP peptides, salt solutions, and mixtures thereof.

10. A method according to claim 1, further comprising extracting whole blood from a human or animal subject in the presence of at least agent selected from the group consisting of anticoagulants, glycosylated peptides, proline-rich peptides, detergents, platelet activators, and mixtures thereof.

11. A method according to claim 10, wherein the agent comprises a platelet activator selected from the group consisting of thrombin and collagen.

12. A method according to claim 10, wherein the agent comprises an anticoagulant are selected from the group consisting of lithium heparin, ammonium heparin, sodium heparin, ethylene diamine tetraacetic acid, acid citrate dextrose, sodium citrate, citrate phosphate dextrose, sodium fluoride, sodium oxalate, potassium oxalate, lithium oxalate, sodium iodoacetate, lithium iodoacetate, and mixtures thereof.

13. A kit comprising:
a heparin-conjugate immobilized on a substrate; and
a device to withdraw whole blood from a human or animal subject.

14. A kit according to claim 13, wherein the substrate is selected from the group consisting of beads, columns, and plates.

15. A kit according to claim 14, wherein the heparin-conjugate is immobilized to the surface by adsorption.

16. A kit according to claim 15, wherein the heparin-conjugate immobilized to the surface of the substrate is fabricated by adsorbing heparin to a polystyrene surface.

17. A kit according to claim 13, further comprising at least one agent selected from the group consisting of anticoagulants, glycosylated peptides, proline-rich peptides, detergents, and platelet activators.

18. A kit according to claim 13, further comprising whole blood.

19. A method of promoting tissue health, comprising:
contacting whole blood with a heparin-conjugated system to separate the whole blood into a conjugated fraction and a non-conjugated fraction;
removing the non-conjugated fractions to provide growth factors; and
applying the growth factors to the tissue.

20. A method of promoting tissue health according to claim 18, wherein the tissue surrounds a wound.

21. A method of promoting tissue health according to claim 18, wherein promoting tissue health further comprises layering the growth factors on an implant.

22. A method of promoting tissue health according to claim 18, wherein the tissues are selected from bone or cartilage.

23. A method of promoting tissue health according to claim 21, further comprising adding osteogenic proteins to the bone or cartilage.
